(19) United States
(12) Patent Application Publication
(10) Pub. No.: US 2025/0331874 A1
(43) Pub. Date: Oct. 30, 2025

(54) SYSTEMS AND METHODS FOR RESTORING BLOOD VESSEL PATENCY

(71) Applicant: Covidien LP, Mansfield, MA (US)

(72) Inventors: Danyong Zeng, Aliso Viejo, CA (US); Ravi Sadasivuni, Aliso Viejo, CA (US); Sydney L. Ngo, Riverside, CA (US); Onnik E. Tchulluian, Costa Mesa, CA (US)

(21) Appl. No.: 19/189,431

(22) Filed: Apr. 25, 2025

Related U.S. Application Data
(60) Provisional application No. 63/639,191, filed on Apr. 26, 2024.

(51) Int. Cl. A61B 17/22

(52) U.S. Cl. CPC A61B 17/22; A61B 2017/22094

(57) ABSTRACT

Devices, systems, and methods for restoring or improving patency of bodily lumens by removing obstructions are disclosed herein. Various embodiments of the present technology are directed to a treatment system comprising an elongated shaft and a treatment device comprising a manipulation member and an interventional element at a distal portion of the manipulation member. In some embodiments, the interventional element comprises a resilient wire formed into a coil having a plurality of longitudinally adjacent windings. The interventional element can be configured to transition between a radially compressed configuration in which the windings are circumferentially aligned and radially expanded configuration in which the windings are circumferentially offset.

### CROSS-REFERENCE TO RELATED APPLICATIONS

This application claims the benefit of and priority to U.S. Provisional Patent Application Ser. No. 63/639,191 filed Apr. 26, 2024, the entire disclosure of which is incorporated by reference herein.

### TECHNICAL FIELD

The present technology relates generally to devices and methods for improving or restoring patency of bodily lumens, for example by removing obstructions from bodily lumens. Some embodiments of the present technology relate to devices and methods for removal of obstructions such as emboli, thrombi, and/or blood clots from blood vessels.

### BACKGROUND

Obstructions within bodily lumens can occlude and/or disrupt the function of the lumen. For example, obstructions such as emboli, thrombi, clots, etc. can occlude blood vessels and interfere with blood flow through vessel lumens. Disruption of blood flow can prevent oxygen and nutrients from being delivered to tissues downstream of the obstruction, which can hinder the tissue from functioning adequately and may result in cellular death. The severity of cellular death increases with duration of occlusion of the vessel.

Ischemic stroke is the result of an obstruction, such as a blood clot, reducing blood flow in a cerebral blood vessel, leading to dysfunction of brain tissue supplied by the cerebral blood vessel. A variety of approaches exist for treating patients experiencing an ischemic stroke. For example, a clinician may administer thrombolytic agents (e.g., tissue plasminogen activator (tPA)) to break down a blood clot occluding a blood vessel. However, there is a limited window in which thrombolytic agents can be administered following stroke onset. Further, thrombolytic agents such as tPA have limited efficacy in treating large vessel occlusions and may cause adverse events if improperly administered to a patient experiencing a hemorrhagic stroke. Ischemic stroke can also be treated with mechanical thrombectomy, an interventional procedure in which a blood clot is removed from a blood vessel using endovascular devices. Mechanical thrombectomy procedures have a longer administration window and can be more effective than thrombolytic agents alone in some cases. Common mechanical thrombectomy techniques include aspirating the blood clot from the blood vessel into a catheter and/or retrieving the blood clot from the blood vessel with a stentriever.

### SUMMARY

Stentrievers are commonly used in mechanical thrombectomy procedures to treat large vessel occlusion (LVO) strokes in which the large, proximal arteries of the brain are occluded by an obstruction such as a thrombus or an embolus. Stentrievers often comprise tubular stents or braids that are radially compressed into a catheter for navigation to the occlusion site. Once the catheter and stentriever have been navigated to the occlusion site and into the obstruction, the stentriever is released from the catheter and radially expands into contact with the obstruction. This contact secures the obstruction to the stentriever so that the stentriever and obstruction can be withdrawn proximally into the lumen of the catheter. The catheter, stentriever, and obstruction are then removed from the body.

As a stentriever radially expands to engage an obstruction, it exerts a radial force on the wall of the vessel, ensuring that the stentriever reaches portions of the obstruction at the wall of the vessel. Consequently, the stentriever also exerts friction on the vessel wall as the stentriever and secured obstruction are drawn proximally into the catheter lumen. The forces exerted on the vessel wall by the stentriever during deployment and retraction can cause endothelial injury and, in severe cases, perforation of the vessel. Laser-cut stents or braids typically apply radial force on vessels to capture or contain obstructions, while also adding lateral stiffness to the vessels, which can stress the vessels and cause adverse events, such as subacute hemorrhagic hematoma (sACH). These risks increase when treating medium vessel occlusions (MVO) in which the obstruction is located in distal vessels that have a smaller diameter (e.g., between about 0.75 mm to about 2 mm, etc.), are more tortuous, and are more flexible and have less structurally support than the large, proximal vessels involved in LVO strokes. The The risks of vessel damage and adverse events with tubular stentrievers are also greater when treating multiple large vessel occlusion (MLVO) strokes involving vessels with different diameters if the stentriever is sized based on the larger, more proximal occluded vessel.

Embodiments of the present technology are directed to devices, systems, and methods for restoring or improving patency of a bodily lumen that address the foregoing challenges and risks. Specifically, treatment devices of the present technology can be configured to apply significantly less radial force and less lateral stiffness to the wall of the bodily lumen than tubular stentrievers. Devices, systems, and methods of the present technology can be configured for treating ischemic stroke, including LVO stroke, MLVO stroke, and/or MEVO stroke. The present technology may be particularly advantageous for treating obstructions in vessels having diameters between about 0.75 mm and about 2 mm, for example in MEVO stroke. Still, the devices, systems, and methods of the present technology can be configured for treating any occluded bodily lumen within any human body systems, such as the peripheral vasculature, the pulmonary vasculature, the coronary vasculature, the gastrointestinal organs, and others.

The present technology is illustrated, for example, according to various aspects described below. Various examples of aspects of the present technology are described as numbered examples for convenience. These are provided as examples and do not limit the present technology. It is noted that any of the dependent examples may be combined in any combination and/or placed into respective independent examples. The other examples can be presented in a similar manner.

### EXAMPLE 1

A method for removing an obstruction from a bodily lumen, the method comprising: obtaining a treatment system comprising an elongated shaft defining a lumen extending therethrough and a treatment device comprising a manipulation member and an interventional element at a distal portion of the manipulation member, the interventional element comprising a resilient wire formed into a coil having a plurality of windings adjacent to one another along a longitudinal dimension of the interventional element, wherein the interventional element is positioned within the lumen of the elongated shaft at a distal portion of the elongated shaft in a radially compressed configuration in which the windings are circumferentially aligned with one another about the longitudinal dimension; distally advancing the distal portion of the elongated shaft through the bodily lumen to a treatment location at or adjacent to the obstruction; releasing the interventional element from the lumen of the elongated shaft such that the interventional element transitions from the radially compressed configuration to a radially expanded configuration in which the windings are circumferentially offset from one another about the longitudinal dimension; engaging the obstruction with the interventional element in the radially expanded configuration; and proximally withdrawing the manipulation member to proximally withdraw the interventional element and the engaged obstruction into the lumen of the elongated shaft.

### EXAMPLE 2

The method of Example 1, wherein each of the windings is defined by a perimeter comprising a first region and a second region, and wherein each of the first regions is closer to the central longitudinal axis in the radially compressed state than in the radially expanded state and each of the second regions is farther from the central longitudinal axis in the radially compressed state than in the radially expanded state.

### EXAMPLE 3

The method of Example 2, wherein the first regions are configured to move away from the central longitudinal axis and the second regions are configured to move towards the central longitudinal axis as the interventional element transitions from the radially compressed state to the radially expanded state.

### EXAMPLE 4

The method of Example 2 or Example 3, wherein each of the first regions and each of the second regions extends circumferentially about the central longitudinal axis.

### EXAMPLE 5

The method of any one of Examples 2 to 3, wherein, when the interventional element is in the radially expanded state, all or a portion of the first region of one of the windings does not circumferentially overlap the first region of another of the windings.

### EXAMPLE 6

The method of any one of Examples 1 to 5, wherein the plurality of windings comprises three windings angularly offset from one another about a circumference of the interventional element by about 120 degrees.

### EXAMPLE 7

The method of any one of Examples 1 to 5, wherein the plurality of windings comprises four windings angularly offset from one another about a circumference of the interventional element by about 90 degrees.

### EXAMPLE 8

The method of any one of Examples 1 to 5, wherein the plurality of windings comprises five windings angularly offset from one another about a circumference of the interventional element by about 72 degrees.

### EXAMPLE 9

The method of any one of Examples 1 to 8, wherein the resilient wire forms the manipulation member.

### EXMPLE 10

The method of any one of Examples 1 to 9, wherein each of the windings defines an opening, and wherein the manipulation member extends through the opening of at least one of the windings.

### EXAMPLE 11

A method for removing an obstruction from a bodily lumen, the method comprising: obtaining a treatment system comprising an elongated shaft defining a lumen extending therethrough and a treatment device comprising a manipulation member and an interventional element at a distal portion of the manipulation member, the interventional element comprising a resilient wire formed into a coil having a first winding and a second winding adjacent the first winding along a central longitudinal axis of the interventional element, wherein the interventional element is positioned within the lumen of the elongated shaft at a distal portion of the elongated shaft in a radially compressed configuration in which the first and second windings are centered about the central longitudinal axis; distally advancing the distal portion of the elongated shaft through the bodily lumen to a treatment location at or adjacent to the obstruction; releasing the interventional element from the lumen of the elongated shaft such that the interventional element transitions from the radially compressed configuration to a radially expanded configuration in which the first and second windings are eccentrically disposed about the central longitudinal axis; engaging the obstruction with the interventional element in the radially expanded configuration; and proximally withdrawing the manipulation member to proximally withdraw the interventional element and engaged obstruction into the lumen of the elongated shaft.

### EXAMPLE 12

The method of Example 11, wherein each of the first and second windings has a first length and second length diametrically opposed to the first length, and wherein, the first lengths are positioned further from the central longitudinal axis in the radially expanded configuration than in the radially compressed configuration and the second lengths are positioned closer to the central longitudinal axis in the radially expanded configuration than in the radially compressed configuration.

### EXAMPLE 13

The method of Example 12, wherein, when the interventional element is in the radially expanded configuration, the first lengths are positioned further from the central longitudinal axis than the second lengths.

### EXAMPLE 14

The method of Example 12 or Example 13, wherein, when the interventional element is in the radially expanded configuration, a maximum outer diameter of the interventional element defined between the first length of the first winding and the first length of the second winding is greater than a local outer diameter of the interventional element defined at any given location along the central longitudinal axis.

### EXAMPLE 15

The method of Example 12 or Example 13, wherein, when the interventional element is in the radially expanded configuration, a maximum outer diameter of the interventional element defined between the first length of the first winding and the first length of the second winding is greater than at least one of a first radial distance defined between the first and second lengths of the first winding or a second radial distance defined between the first and second lengths of the second winding.

### EXAMPLE 16

The method of any one of Examples 12 to 15, wherein at least one of the first length of the first winding or the first length of the second winding has a greater radius of curvature than the second length of the respective winding.

### EXAMPLE 17

The method of any one of Examples 12 to 15, wherein at least one of the first length of the first winding or the first length of the second winding has a greater arc length than the second length of the respective winding.

### EXSMPLE 18

The method of any one of Examples 11 to 17, wherein the resilient wire forms the manipulation member.

### EXAMPLE 19

The method of any one of Examples 11 to 18, wherein each of the first and second windings defines an opening, and wherein the manipulation member extends through the openings of the first and second windings.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a perspective view of a manipulation member and an interventional element in an expanded configuration in accordance with the present technology.

FIG. 1B is a perspective view of the manipulation member and interventional element of FIG. 1A in a compressed configuration within a lumen of an elongated shaft in accordance with the present technology.

FIGS. 1C and 1D are isolated perspective and end views, respectively, of the interventional element of FIG. 1A.

FIG. 1E is an isolated view of one end element and one winding of the interventional element of FIGS. 1A-1D.

FIGS. 2A and 2B schematically depict a method of improving or restoring patency of a bodily lumen with a treatment system in accordance with the present technology.

FIGS. 3-7 depict various interventional elements in accordance with the present technology.

FIG. 8 is a perspective view of a manipulation member and interventional elements in an expanded configuration in accordance with the present technology.

FIG. 9 is a side schematic view of a manipulation member and interventional elements in an expanded configuration in accordance with the present technology.

### DETAILED DESCRIPTION

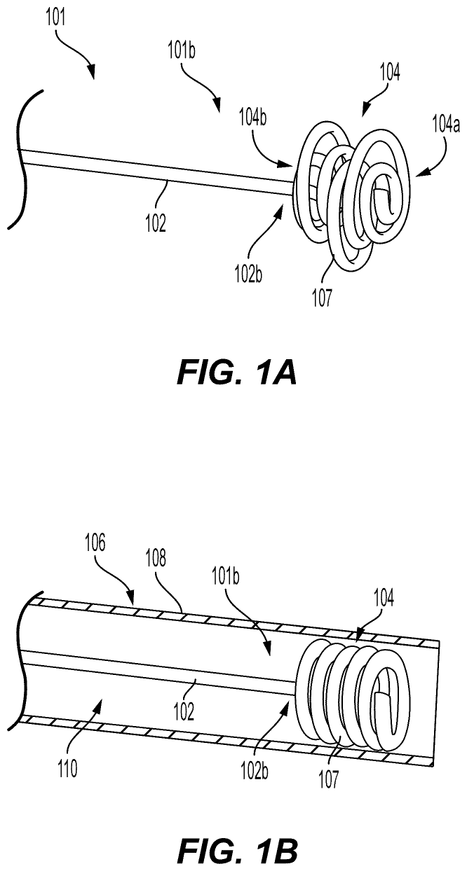

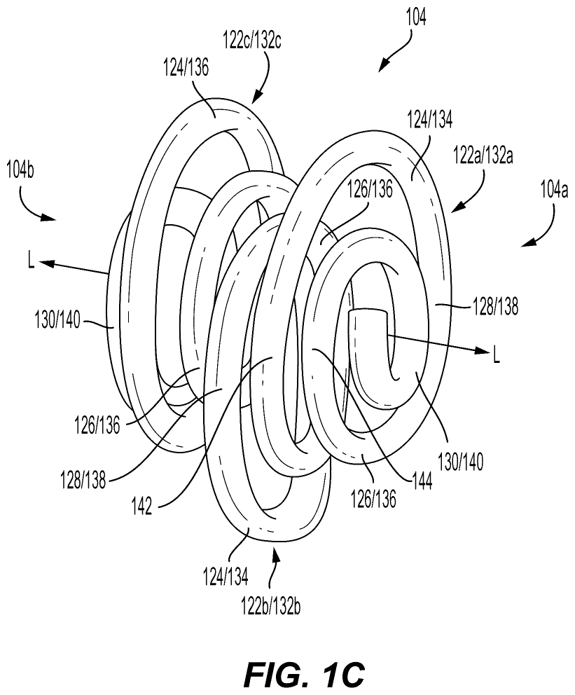

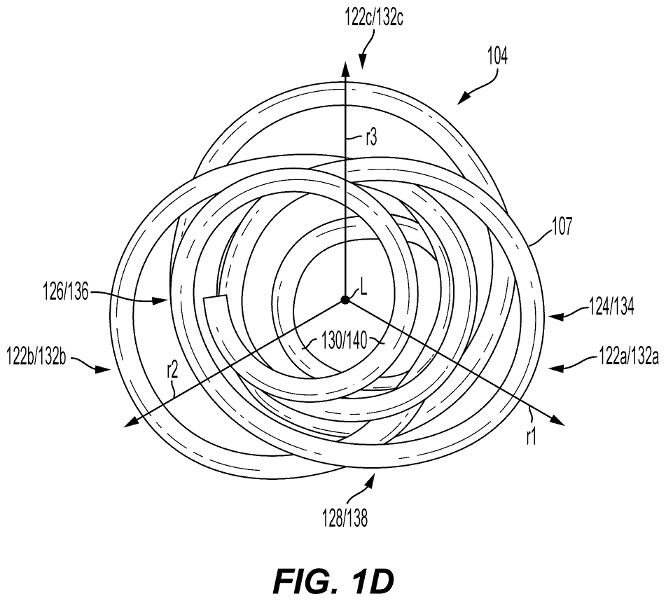

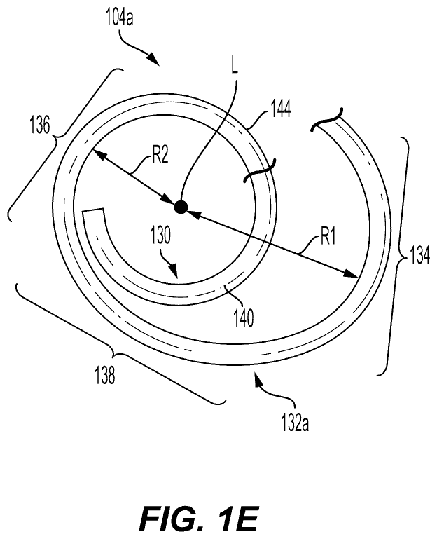

The present technology relates to devices, systems, and methods for improving or restoring patency of a bodily lumen by treating an obstruction at an occlusion site within the bodily lumen. Treating the obstruction often includes removing all or a portion of the obstruction from the occlusion site and/or otherwise modifying the shape of the obstruction to improve patency within the bodily lumen. The treatment systems disclosed herein comprise an elongated shaft and a treatment device including a manipulation member and an interventional element. The interventional element can comprise a resilient wire formed into a coil, and is carried by, coupled to, and/or continuous with a distal portion of the manipulation member. The manipulation member and interventional element are configured to be slidably received within a lumen of the elongated shaft with the interventional element in a radially constrained, compressed configuration. When the interventional element is in an expanded configuration, adjacent windings of the coil extend away from a longitudinal axis of the coil in different directions, thereby applying significantly less radial force to the wall of the bodily lumen than traditional, constant-diameter stentrievers. Once engaged with the obstruction, the interventional element and manipulation member can be proximally drawn through the bodily lumen and, optionally, into the lumen of the elongated shaft to remove the obstruction from the occlusion site and, ultimately, from the bodily lumen. In some embodiments, aspiration can be used in conjunction with the interventional element to remove the obstruction. Specific details of several embodiments of the technology are described below with reference to FIGS. 1A-9.

FIG. 1A depicts a distal portion 101b of a treatment device 101 (also referred to as “device 101”) for restoring or improving patency of a bodily lumen in accordance with various embodiments of the present technology. The device 101 includes a manipulation member 102 and an interventional element 104 at a distal portion 102b of the manipulation member 102. The interventional element 104 can have an expanded, unconstrained configuration (as shown in FIG. 1A) for engaging clot material and a radially constrained, compressed configuration when positioned within the lumen 110 of an elongated shaft 106 (as shown in FIG. 1B) during intravascular delivery to a treatment site. The interventional element 104 can comprise a coil formed of an elongated element 107 wound around a central longitudinal axis L to form a series of windings. The interventional element 104 and/or elongated element 107 can comprise a resilient material such that the interventional element 104 is configured to transition from the compressed configuration to the expanded configuration when released from the constraint of the elongated shaft 106. As discussed in greater detail below, at least some of the windings of the coil can be eccentrically disposed about the central longitudinal axis L such that said windings extend away from the longitudinal axis L in different radial directions. As a result, the interventional element 104 has a varying outer diameter that applies significantly less radial force to the wall of the bodily lumen than traditional, constant-diameter stentrievers.

The manipulation member 102 can comprise an elongated member extending from a proximal portion (not shown) to a distal portion 102b along a longitudinal axis of the device 101. The elongated member can be a wire, a tube, a coil, a braid, and/or other suitable structures. A diameter of the manipulation member 102 may vary and/or taper along some or all of its length. The manipulation member 102 may include one or more fluorosafe and/or radiopaque markers (not shown) comprising a band, a deposited material, an exposed portion of the manipulation member 102, etc.

FIGS. 1C and 1D show isolated perspective and end views, respectively, of the interventional element 104. Referring to FIGS. 1A-1D together, the interventional element 104 has a first end portion 104a, a second end portion 104b, and a central longitudinal axis L extending therebetween. The second end portion 104b can be secured to and/or continuous with the manipulation member 102. The first end portion 104a can be secured to the manipulation member 102 or may comprise a free end (as shown in FIG. 1A) configured to move independently of the manipulation member 102.

In some embodiments, for example as shown in FIGS. 1A and 1B, the interventional element 104 is positioned at a distal terminus of the manipulation member 102 such that the manipulation member 102 does not extend distally beyond the interventional element 104. Alternatively, the manipulation member 102 can extend distally beyond the interventional element 104 with the interventional element 104 proximal of the distal terminus of the manipulation member 102. In any case, the interventional element 104 is configured to move with the manipulation member 102, i.e., rotation of manipulation member 102 causes a corresponding rotation of the interventional element 104 and translation of the manipulation member 102 causes a corresponding translation of the interventional element 104. In other embodiments the interventional element 104 can be coupled to the manipulation member 102 such that the interventional element 104 can rotate independently of the manipulation member 102. The proximal portion of the manipulation member 102 can be configured to be manipulated by an operator (e.g., a clinician, a robotic actuator, etc.) to move the interventional element 104 through the lumen 110 of the elongated shaft 106 and a bodily lumen.

In some embodiments the interventional element 104 is a structure separate from that of the manipulation member 102 and is coupled to a distal portion of the manipulation member 102. For example, the interventional element 104 can be formed of a separate elongated element 107 wound into a coil. The elongated element 107 can comprise a wire, a tube, a coil, a braid, and/or other suitable elongated structures. At least a proximal end portion of the elongated element 107 can be coupled to the distal end portion 102b of the manipulation member 102. In these and other embodiments, the manipulation member 102 can be positioned through one or more openings formed by the windings of the coil. According to some embodiments, the interventional element 104 is monolithic with the manipulation member 102 such that the distal portion of the manipulation member 102 forms the elongated element 107 of the coil.

All or some of the windings of the interventional element 104 can individually define engagement elements 122 (labeled and referred to individually as first engagement element 122a, second engagement element 122b, and third engagement element 122c) configured to contact, secure to, enmesh with, embed within, capture, fragment, or otherwise engage and/or modify an obstruction within a bodily lumen. As best visualized in FIG. 1D, the engagement elements 122 extend away from the central longitudinal axis L in different radial directions (r1, r2, r3) when the interventional element 104 is in the expanded configuration. As such, the engagement elements 122 are eccentrically disposed about the central longitudinal axis L and longitudinally offset relative to one another.

At least when the interventional element 104 is in the expanded configuration, the engagement elements 122 can be offset from one another about a circumference of the interventional element 104. Each engagement element 122 or corresponding winding can be angularly spaced apart from one or more longitudinally adjacent engagement elements 122 or windings by about 180 degrees, about 150 degrees, about 120 degrees, about 90 degrees, about 72 degrees, about 60 degrees, about 30 degrees, between about 30 degrees and about 180 degrees, between about 60 degrees and about 150 degrees, or between about 90 degrees and about 120 degrees. When the interventional element 104 is in the compressed configuration, the engagement elements 122 can be circumferentially offset from one another to a lesser degree and/or can be circumferentially aligned.

Although FIGS. 1A-1D illustrate three engagement elements 122, the interventional element 104 can include more or fewer engagement elements 122 (e.g., one engagement element 122, two engagement elements 122, four engagement elements 122, five engagement elements 122, etc.).

Each of the windings comprising the individual engagement elements 122 include a first region 124 and a second region 126 diametrically opposed to the first region 124. Each of the windings comprising an engagement element 122 can further comprise a third region 128 extending between the first region 124 and the second region 126. Each of the first regions 124 and each of the second regions 126 can extend in a circumferential direction about the central longitudinal axis L of the interventional element 104. In some embodiments, the first region 124 of a given winding can have a greater radius of curvature and/or arc length than the second region 126 of the given winding at least when the interventional element 104 is in the unconstrained, expanded configuration. Still, in some embodiments, the first and second regions 124, 126 can have the same radius of curvature and/or arc lengths. In various embodiments, one or more of the engagement elements 122 are eccentrically shaped such that at least one portion of the engagement element (or winding) is farther from the longitudinal axis L than another portion of the engagement element (or winding).

When the interventional element 104 is in the expanded configuration, for example as shown in FIGS. 1A, 1C and 1D, the first region 124 of each engagement element 122 can be farther from the central longitudinal axis L than when the interventional element 104 is in the compressed configuration (see FIG. 1B), and the second region 126 of each engagement element 122 can be closer to the central longitudinal axis L than when the interventional element 104 is in the compressed configuration. Thus, when the interventional element 104 is positioned within the lumen 110 of the elongated shaft 106, the sidewall 108 of the elongated shaft 106 can, at least initially, contact only a portion of each engagement element 122, which can cause the engagement elements 122 to shift relative to one another and the central longitudinal axis L. Shifting rather than deforming the engagement elements 122 under the compression of the sidewall 108 can maintain a desired elasticity and resilience of the engagement elements 122. Still, in some embodiments, one or more portions of the interventional element 104 can rotate and/or deform, in addition to or instead of shifting, in response to compression by the sidewall 108 of the elongated shaft 106.

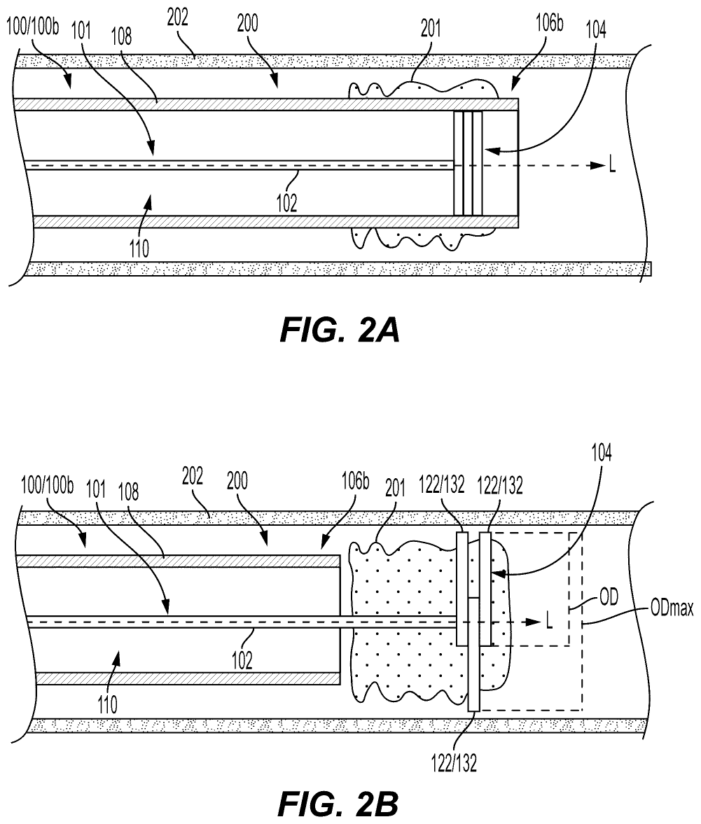

In any case, the maximum outer diameter ODmax of the interventional element 104 (measured between the radially outermost portions of the engagement elements 122, as labeled in FIG. 2B) in the compressed configuration can be less than or equal to a diameter of the lumen 110 of the elongated shaft 106. In some embodiments, the maximum outer diameter ODmax can be substantially constant in the compressed configuration. Likewise, the engagement elements 122 can be substantially centered about the central longitudinal axis L when the interventional element 104 is in the compressed configuration. When the interventional element 104 is released from the compressed configuration and assumes the expanded configuration, the first regions 124 can move away from the central longitudinal axis L and the second regions 126 can move towards the central longitudinal axis L, thereby increasing the maximum outer diameter ODmax of the interventional element 104.

In some embodiments, for example as shown in FIGS. 1A-1D, the first engagement element 122a can comprise a first winding 132a, the second engagement element 122b can comprise a second winding 132b, and/or the third engagement element 122c can comprise a third winding 132c (collectively “windings 132”). FIG. 1E shows an isolated view of the first winding 132a and one of the end elements 130 of the interventional element 104 of FIGS. 1C and 1D (e.g., in an expanded configuration). Only the first winding 132a is shown in FIG. 1E for ease of illustration, but any of the windings 132 can have similar features to the first winding 132a. As shown in FIG. 1E, the first winding 132a can comprise a first length 134 of the elongated element 107, a second length 136 of the elongated element 107 opposed to the first length 134 about a circumference of the first winding 132a, and a third length 138 of the elongated element 107 extending between the first length 134 and the second length 136. The first length 134 can correspond to the first region 124 of the engagement element 122, the second length 136 can correspond to the second region 126 of the engagement element 122, and/or the third length 138 can correspond to the third region 128 of the engagement element 122. In some embodiments, the end element 130 comprises a fourth length 140 of the elongated element 107.

The windings 132 and/or the end elements 130 can be longitudinally offset from one another. In some embodiments, a second end of a winding 132 and/or end element 130 can be continuous with the first end of an adjacent winding 132 and/or end element 130. In these embodiments, and others, only a portion of the winding 132 and/or end element 130 may be longitudinally offset from a corresponding portion of the adjacent winding 132 and/or end element 130. For example, the first lengths 134 of adjacent windings 132 can be longitudinally offset from one another. In some embodiments, the lengths of the elongated element 107 forming one or more of the windings 132 and/or end elements 130 extend only circumferentially, so that the winding 132 and/or end element 130 has a single position along the central longitudinal axis L of the interventional element 104. In various embodiments, the interventional element 104 can include one or more lengths of elongated element 107 and/or loops between longitudinally adjacent windings 132 and/or end elements 130. For example, the interventional element 104 can include a fifth length 142 between a first length 134 of one winding 132 and the second length 136 of a sequential winding 132 and/or a sixth length 144 between a fourth length 140 of an end element 130 and a length of an adjacent winding 132. Only the fifth length 142 between the first length 134 of the first winding 132a and the second length 136 of the second winding 132b and the sixth length 144 between the fourth length 140 of the end element 130 at the first end portion 104a and the second length 136 of the first winding 132a are labeled in FIG. 1C for ease of illustration. One, some, or all of the fifth lengths 142 and/or the sixth lengths 146 can extend in a circumferential direction about the central longitudinal axis L of the interventional element 104. One, some, or all of the fifth lengths 142 and/or the sixth lengths 146 can extend along the central longitudinal axis L of the interventional element 104.

According to various embodiments, adjacent windings 132 can be circumferentially offset from one another about the central longitudinal axis L in at least the expanded configuration such that all or a portion of a first length 134 of one of the windings 132 does not overlap a first length 134 of an adjacent one of the windings 132. The first lengths 134 of adjacent windings 132 can be located at different circumferential positions in at least the expanded configuration. Said another way, in at least the expanded configuration, the windings 132 can extend from the central longitudinal axis L in different radial directions. As discussed with reference to the engagement elements 122, the windings 132 can be eccentrically disposed about the central longitudinal axis L in the expanded configuration. In the compressed configuration, the windings 132 can be centered about the central longitudinal axis L and/or offset about the central longitudinal axis L to a lesser degree than in the expanded configuration.

The second length 136 of a winding 132 can be diametrically opposed to the first length 134 of the winding 132. In some embodiments, the second length 136 is angularly spaced apart from the first length 134 about the central longitudinal axis L by about 180 degrees. In some embodiments, the second length 136 can be angularly spaced apart from the first length 134 about the central longitudinal axis L by between about 30 degrees to about 330 degrees, between about 60 degrees to about 300 degrees, between about 90 degrees to about 270 degrees, between about 120 degrees to about 240 degrees, or between about 150 degrees to about 210 degrees.

The first length 134 can have a first radius of curvature and the second length 136 can have a second radius of curvature different from the first radius of curvature. In some embodiments, for example as shown in FIG. 1E, the second radius of curvature is less than the first radius of curvature. Each of the first length 134 and the second length 136 can extend circumferentially about the central longitudinal axis L of the interventional element 104 from a first end to a second end. An arc length of the first length 134 can be greater than arc length of the second length 136. The first length 134 can be radially spaced apart from the central longitudinal axis L by a first radial distance R1 and the second length 136 can be radially spaced apart from the central longitudinal axis L by a second radial distance R2. At least when the interventional element 104 is in the expanded configuration, the first radial distance R1 can be greater than the second radial distance R2. At least when the interventional element 104 is in the expanded configuration, the third length 138 can extend radially outwardly from the second length 136 to the first length 134. One, some, or all of the fifth lengths 142 and/or the sixth lengths 146 can have a radius of curvature, an arc length, and/or a radial distance from the central longitudinal axis L between the corresponding parameters of the lengths of elongated element 107 to which the fifth or sixth length connects.

Because of the eccentric geometry of the first winding 132a and/or the eccentric positioning of the first winding 132a about the central longitudinal axis L in the expanded configuration, when the interventional element 104 is positioned within the lumen 110 of the elongated shaft 106 and forced to assume the compressed configuration, the first length 134 may initially engage the inner surface of the sidewall 108 of the elongated shaft 106 while the second length 136 does not initially engage the inner surface of the sidewall 108 of the elongated shaft 106. Thus, inner surface of the sidewall 108 of the elongated shaft 106 may (at least initially) apply radially compressive forces to the first length 134 but not the second length 136. Such forces may push the first length 134 closer to the central longitudinal axis L of the interventional element 104, which can cause the second length 136 to move away from the central longitudinal axis L of the interventional element 104. In other words, the first radial distance R1 can decrease while the second radial distance R2 increases. In this manner, the first winding 132a can be radially shifted with respect to the central longitudinal axis L when in the compressed configuration as compared to the expanded configuration. Moreover, because radial compression is not initially applied to the entire circumference of the first winding 132a, the first winding 132a is able to shift rather than deform under the compression. By preventing and/or limiting deformation of the first winding 132a, the first winding 132a can maintain its elasticity and resilience, which can facilitate expansion of the interventional element from the compressed configuration upon release from the lumen 110 of the elongated shaft 106.

As shown in FIGS. 1C-1E, for example, the fourth length 140 of elongated element 107 forming the end element 130 may comprise one complete loop (e.g., extending about the central longitudinal axis L by 360 degrees), less than one complete loop (e.g., extending about the central longitudinal axis L by less than 360 degrees), or more than one complete loop (e.g., extending about the central longitudinal axis L by more than 360 degrees). The fourth length 140 forming the end element 130 can have a substantially constant radius of curvature or a varying radius of curvature. The fourth length 140 can be continuous, unitary, and/or monolithic with the elongated element 107 forming an adjacent winding 132 of the interventional element 104. In some embodiments, one or more end elements 130 does not comprise a wound elongated element 107 and instead comprises a disc, a ring, a nut, a tube, a melted material, a glue, or another structure that facilitates termination of the windings 132 of the interventional element 104 and/or attachment of the interventional element 104 to the manipulation member 102. In some embodiments, the interventional element 104 does not include an end element 130 at its first end portion 104a and/or at its second end portion 104b.

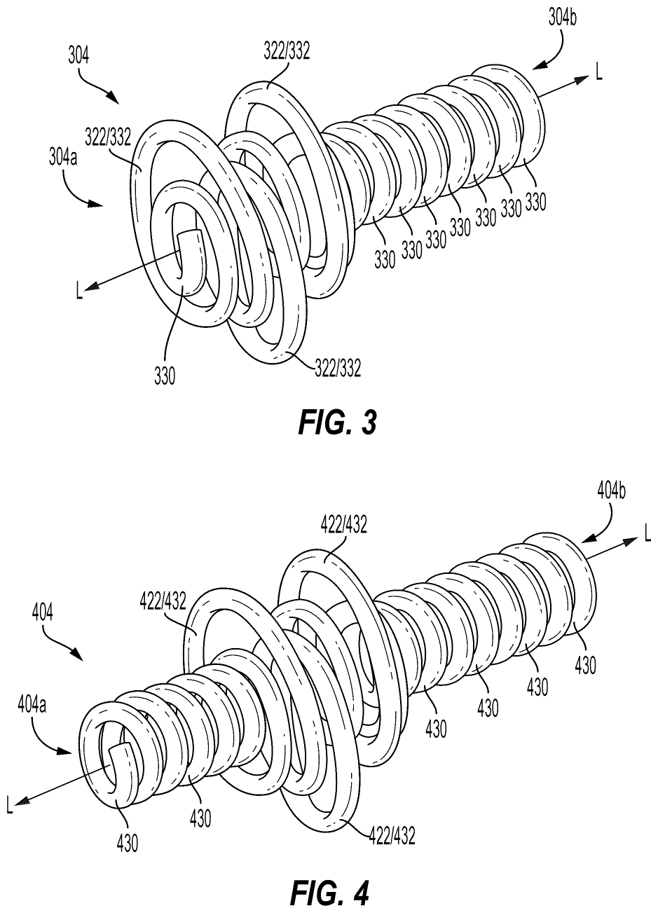

As previously noted, in some embodiments, one or more of the end elements 130 can be configured to facilitate attachment of the interventional element 104 to the manipulation member 102. Such an end element 130 can be configured to be secured to the manipulation member 102 and/or continuous, unitary, and/or monolithic with the manipulation member 102. One or more of the end elements 130 can define an opening configured to receive the manipulation member 102 to mount the interventional element 104 on the manipulation member 102. The end element 130 at the first end portion 104a of the interventional element 104 and/or the end element 130 at the second end portion 104b of the interventional element 104 can be configured to be fixed to the manipulation member 102. For example, the end element(s) 130 can be soldered, welded, melted, mechanically joined, adhered, glued, or otherwise fixed to the manipulation member 102. In these embodiments, and others, the end element(s) 130 can be configured to move longitudinally and rotationally with the manipulation member 102. In some embodiments, the end element(s) 130 can be longitudinally constrained relative to the manipulation member 102 but configured to rotate over the manipulation member 102. For example, the manipulation member 102 can carry one or more stops (not shown) that are fixed to the manipulation member 102 and, when the interventional element 104 is mounted on the manipulation member 102, the stop(s) prevent or limit longitudinal motion of the interventional element 104 relative to the manipulation member 102. In some embodiments, for example as shown in FIGS. 3-7, a plurality of end elements 130 can extend proximally from the windings 132 of the interventional element 104 to form an elongated coil. In these embodiments, and others, the end elements 130 can form the manipulation member 102 of the device 101. Additionally or alternatively, a plurality of end elements 130 can extend distally from the windings 132 of the interventional element 104 to form an elongated coil.

The end elements 130 can have outer diameters smaller than the outer diameters of the windings 132 at least when the interventional element 104 is in the expanded configuration. In some embodiments, the end elements 130 can have outer diameters larger than or approximately equal to an outer diameter of the manipulation member 102. A diameter of an end element 130 can be slightly larger than an outer diameter of the manipulation member 102 but smaller than an outer diameter of an adjacent winding 132 to provide a smooth transition between the manipulation member 102 and the winding 132 of the interventional element 104.

In embodiments in which the manipulation member 102 and the interventional element 104 comprise a wire, the manipulation member 102 and the interventional element 104 can be formed from a single wire or from multiple wires that are joined together. A wire used to form the manipulation member 102 and/or the interventional element 104 can comprise any metal, polymer, or other biocompatible material. In some embodiments, the material of a wire used to form the interventional element 104 is based on a desired resilience of the interventional element 104. For example, it may be desirable for the material to be able to withstand a predetermined amount of strain without yielding. A wire used to form the manipulation member 102 can have sufficient stiffness for the manipulation member 102 to support longitudinal motion of the interventional element 104 through the lumen 110 of the elongated shaft 106 and/or while engaging and moving an obstruction within a bodily lumen. In some embodiments, the wire comprises stainless steel, nickel cobalt (e.g., MP35N), Nitinol, platinum, alloys thereof, and/or other materials. The wire can comprise a core material with one or more additional materials carried by the core material. For example, the wire can comprise a drawn-filled tube. In some embodiments, the wire can include a core material and/or an outer material that is radiopaque to facilitate visualization of the manipulation member and/or interventional element 104. Additionally or alternatively, the wire can include a core material carrying a polymeric outer material that is hydrophobic.

A wire used to form the manipulation member 102 and/or the interventional element 104 can have a diameter that is substantially constant or variable along its length. The wire can have a distal end that is flat or rounded. The shape of the wire can be configured to facilitate navigation of the manipulation member 102 and/or the interventional element 104 through the elongated shaft lumen and/or the bodily lumen.

FIGS. 2A and 2B schematically illustrate a method of using the treatment system 100 of FIGS. 1A-1E to restore or improve patency of a bodily lumen 200 occluded by an obstruction 201. As shown in FIG. 2A, the distal portion 100b of the system 100 can be navigated through the bodily lumen 200 and positioned at a treatment site that is at or adjacent to the obstruction 201. The distal portion 100b can be navigated through the bodily lumen 200 with the treatment device 101 loaded in the lumen 110 of the elongated shaft 106 and/or the treatment device 101 can be slidably moved through the lumen 110 after the elongated shaft 106 has been navigated through the bodily lumen 200. The proximal portion (not shown) of the manipulation member 102 can be manipulated to move the interventional element 104 through the lumen 110 of the elongated shaft 106 and/or the bodily lumen 200. For example, the proximal portion of the manipulation member 102 can be pulled proximally to move the interventional element 104 proximally, the proximal portion of the manipulation member 102 can be pushed distally to move the interventional element 104 distally, the proximal portion of the manipulation member 102 can be rotated to rotate the interventional element 104, etc.

The distal portion 106b of the elongated shaft 106 and/or the interventional element 104 can be positioned within the bodily lumen 200 at or adjacent to the obstruction 201 with the interventional element 104 within the lumen 110 of the elongated shaft 106 in the compressed configuration. For example, FIG. 2A illustrates the distal portion 106b of the elongated shaft 106 positioned distal of the obstruction 201 such that the elongated shaft 106 extends through the obstruction 201 and the interventional element 104 is positioned within the obstruction 201 within the lumen 110 of the elongated shaft 106. Thus, the elongated shaft 106 can be proximally withdrawn relative to the interventional element 104 such that the interventional element 104 expands into contact with the obstruction 201 (see FIG. 2B). In some embodiments, the distal portion 106b of the elongated shaft 106 and the interventional element 104 can be positioned distal of the obstruction 201 while the interventional element 104 is within the lumen 110 of the elongated shaft 106. In these embodiments, and others, the interventional element 104 can be expanded distal of the obstruction 201. The interventional element 104 can then be drawn proximally into contact with the obstruction 201.

To deploy the interventional element 104, the elongated shaft 106 can be drawn proximally while proximal motion of the manipulation member 102 and thereby the interventional element 104 is prevented or limited. Additionally or alternatively, the manipulation member 102 and thereby the interventional element 104 can be advanced distally while distal motion of the elongated shaft 106 is prevented or limited. Either way, the interventional element 104 is expelled from the lumen 110 of the elongated shaft 106 and expands (see FIG. 2B). The engagement elements 122 of the interventional element 104 shift, rotate, and/or deform when released from the radial constraint of the elongated shaft 106. For example, as previously discussed, the first regions 124 of the engagement elements 122 (e.g., the first lengths 134 of the windings 132, etc.) can move away from the central longitudinal axis L of the interventional element 104 and the second regions 126 of the engagement elements 122 (e.g., the second lengths 136 of the windings 132, etc.) can move towards the central longitudinal axis L such that the engagement elements 122 are eccentrically disposed about the central longitudinal axis L.

In the expanded configuration, the engagement elements 122 can be configured to extend across the bodily lumen 200 and engage the obstruction 201. In some embodiments, the interventional element 104 has a maximum outer diameter ODmax in the expanded configuration substantially equal to or slightly larger than a diameter of the bodily lumen 200 so that the interventional element 104 can engage portions of the obstruction 201 at the wall 202 of the bodily lumen 200. Still, in some embodiments the interventional element 104 has a maximum outer diameter ODmax in the expanded configuration less than a diameter of the bodily lumen 200. As shown in FIG. 2B, at a given location along the central longitudinal axis L, the interventional element 104 can have a local outer diameter OD (measured between radially opposed portions of the coil) that is smaller than the maximum outer diameter of the interventional element 104. Thus, at the given location the interventional element 104 the interventional element 104 may not contact the entire circumference of the wall 202 of the bodily lumen 200. However, the interventional element 104 can include multiple engagement elements 122 that are longitudinally and/or circumferentially offset from one another such that the interventional element 104 as a whole is configured to extend about the entire circumference of the bodily lumen 200 and/or contact the entire circumference of the wall 202. Thus, the interventional element 104 can engage the obstruction 201 regardless of its circumferential position within the bodily lumen 200.

Moreover, each engagement element 122 is configured to contact less than the entire circumference of the wall 202, thereby limiting the outward radial force that each engagement element 122 can apply to the wall 202 and reducing the risk of damaging the wall 202 during deployment of the interventional element 104. Because longitudinally adjacent engagement elements 122 can be attached to one another by flexible wound portions of an elongated element 107, the engagement elements 122 can tilt about the central longitudinal axis L of the interventional element 104. Thus, when the interventional element 104 is moved longitudinally within the bodily lumen 200 (e.g., while retrieving the obstruction, etc.), the engagement elements 122 can tilt if they contact the wall 202 rather than applying longitudinal forces to the wall 202, further reducing the risk of damaging the wall 202 during the treatment. In contrast, typical tubular mechanical thrombectomy devices engage the entire circumference of the wall 202 along their entire length and apply significant radial force to the wall 202 while deploying the device and longitudinal force to the wall 202 while moving the device through the bodily lumen 200.

After being at least partially released from the lumen 110 of the elongated shaft 106, the interventional element 104 (whether secured to the obstruction 201 or not) can be resheathed and withdrawn proximally back into the lumen 110 of the elongated shaft 106. To move the interventional element 104 proximally relative to the elongated shaft 106, the elongated shaft 106 can be moved distally relative to the bodily lumen 200 while distal motion of the manipulation member 102 and thereby the interventional element 104 relative to the bodily lumen 200 is prevented or limited and/or the manipulation member 102 and thereby the interventional element 104 can be moved proximally relative to the bodily lumen 200 while proximal motion of the elongated shaft 106 relative to the bodily lumen 200 is prevented or limited. Upon reentry into the lumen 110, the interventional element 104 can transition from the expanded configuration to the compressed configuration.

Once the interventional element 104 has engaged the obstruction 201, the interventional element 104 and secured obstruction 201 can be withdrawn proximally into the lumen 110 of the elongated shaft 106 (and/or another catheter of the treatment system, such as an aspiration catheter, etc.) and subsequently removed from the body. Additionally or alternatively, the interventional element 104 can be configured to fragment the obstruction 201, crush the obstruction 201 against the wall 202, move the obstruction 201 within the bodily lumen 200, or otherwise modify the obstruction 201 such that the obstruction 201 occludes the bodily lumen 200 to a lesser degree. Notably, as the interventional element 104 is drawn proximally into a lumen into a radially constrained configuration, the longitudinally adjacent windings are sequentially subjected to compression at only a portion of the circumference of each winding. Conversely, traditional stentrievers are compressed around their entire circumference when drawn into a lumen and, as the stentriever is compressed from proximal to distal, the complete circumferential compression can tend to squeeze the obstruction towards and/or past the distal end of the stentriever and back into the bodily lumen.

In some embodiments, the treatment system 100 includes one or more catheters in addition to the elongated shaft 106. For example, the system 100 can include a catheter having a larger inner diameter than the outer diameter of the elongated shaft 106 so that the elongated shaft 106 can longitudinally slide through the lumen of the catheter. In some embodiments, the inner diameter of the catheter can be sized to receive the obstruction 201. During use, a distal end portion of the catheter can be positioned proximal of the obstruction 201 and/or the distal end portion of the elongated shaft 206.

Any of the methods of the present technology can involve aspirating the obstruction 201 in addition to engaging and modifying the obstruction 201 with the treatment device 101. For example, a negative pressure can be created within the lumen 110 of the elongated shaft 106 and/or a separate aspiration catheter can be navigated to the obstruction 201 and a negative pressure can be created within the lumen of the aspiration catheter. The negative pressure can cause the obstruction 201 to move towards the elongated shaft 106 and/or aspiration catheter. In some embodiments, the interventional element 104 and/or the obstruction can be drawn proximally into the aspiration catheter lumen, in addition to or instead of the lumen 110 of the elongated shaft 106 used to deliver the interventional element 104. The negative pressure can be created prior to deployment of the interventional element 104, during deployment of the interventional element 104, or after deployment of the interventional element 104. A pressure source (e.g., a vacuum pump, a syringe, etc.) can be fluidically coupled to the elongated shaft 106 and/or the separate aspiration catheter to create the negative pressure.

FIGS. 3-7 illustrate representative examples of interventional elements 304-704 in accordance with embodiments of the present technology. The features of the interventional elements 304-704 can be generally similar to the features of the interventional element 104 of FIGS. 1A-2B. Accordingly, like numbers (e.g., windings 332 versus windings 132) are used to identify similar or identical components in FIGS. 1A-7, and the discussion of the interventional elements 304-704 of FIGS. 3-7 will be largely limited to those features that differ from the interventional element 104 of FIGS. 1A-2B. Additionally, any of the features of the interventional elements 304-704 of FIGS. 3-7 can be combined with each other and/or with the features of the interventional element 104 of FIGS. 1A-2B. Any of the systems or treatment devices disclosed herein can include any of the interventional elements 304-704 of FIGS. 3-7.

The interventional element 304 shown in FIG. 3 comprises a first end portion 304a, a second end portion 304b, and a central longitudinal axis L extending therebetween. The first end portion 304a can comprise a proximal end portion or a distal end portion of the interventional element 304. Similar to the interventional element 104 of FIGS. 1A-2B, the interventional element 304 of FIG. 3 can comprise a plurality of circumferentially and/or longitudinally offset engagement elements 322. In some embodiments, the interventional element 304 comprises a coil formed from a wire or other elongated element wound about the central longitudinal axis L in a plurality of windings 332, which can correspond to the engagement elements 322. The interventional element 304 can comprise a plurality of end elements 330. For example, as shown in FIG. 3, interventional element 304 can comprise one end element 330 at the first end portion 304a of the interventional element 304 and a plurality of end elements 330 at the second end portion 304b of the interventional element 304. The plurality of end elements 330 at the second end portion 304b can comprise a series of contiguous loops. Diameters of the end elements 330 can be substantially constant along the longitudinal axis L or may vary. Pitches between adjacent end elements 330 can be substantially constant along the longitudinal axis L or may vary.

As previously noted with respect to interventional element 104, interventional element 304 can be configured to be carried by, secured to, and/or monolithic with a manipulation member (such as manipulation member 102, etc.). An endmost end element 330 at either side of the interventional element 304 can be configured to be secured to and/or can be monolithic with a distal end of the manipulation member. In some embodiments, openings defined by the windings 332 and/or the end elements 330 can be configured to receive the manipulation member such that the interventional element 304 is mounted on the manipulation member. The plurality of end elements 330 at the second end portion 304b of the interventional element 304 can facilitate centering of the interventional element 304 on the manipulation member, as a greater number of end elements 330 can better resist movement of the longitudinal axis L of the interventional element 304 away from a manipulation member without substantial deformation of the interventional element 304. Additionally or alternatively, the plurality of end elements 330 can be fixed to the manipulation member to prevent or limit sliding and/or rotation of at least the second end portion 304b of the interventional element 304 relative to the manipulation member. In some embodiments, the manipulation member can carry one or more stops that control the position of the interventional element 304 on the manipulation member.

According to various embodiments, the manipulation member can comprise a wire wound into a coil. In these embodiments, and others, the plurality of end elements 330 can form the manipulation member. The plurality of end elements 330 can be sufficiently long to extend from an extracorporeal position to an intended treatment location within a bodily lumen.

The interventional element 404 shown in FIG. 4 comprises a first end portion 404a, a second end portion 404b, and a central longitudinal axis L extending therebetween. The first end portion 404a can comprise a proximal end portion or a distal end portion of the interventional element 404. The interventional element 404 of FIG. 4 can comprise a plurality of circumferentially and/or longitudinally offset engagement elements 422. In some embodiments, the interventional element 404 comprises a coil formed from a wire or other elongated element wound about the central longitudinal axis L in a plurality of windings 432. The interventional element 404 can comprise a plurality of end elements 430. Similar to the interventional element 304 of FIG. 3, the interventional element 404 of FIG. 4 comprises a plurality of end elements 430 at the second end portion 404b of the interventional element 404 (only a few of which are labeled for ease of illustration). However, instead of a single end element at the first end portion 404a, interventional element 404 comprises a plurality of end elements 430 at the first end portion 404a (only a few of which are labeled for ease of illustration). Providing multiple end elements 430 on both sides of the engagement elements/windings 422/432 can further facilitate centering of the interventional element 404 on a manipulation member extending through the windings of the interventional element 404. Additionally or alternatively, the end elements 430 positioned on either side of the engagement elements/windings 422/432 can be fixed to a manipulation member to prevent or limit sliding and/or rotation of the interventional element 404 relative to the manipulation member. When the end elements 430 on both sides of the engagement elements/windings 422/432 are fixed to a manipulation member, elongation of the interventional element 404 under radial compression can be prevented or limited.

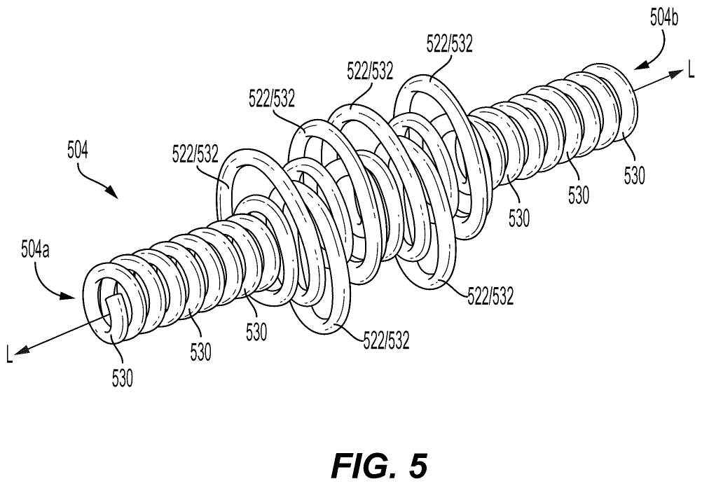

The interventional elements 304, 404 of FIGS. 3 and 4 each have three engagement elements/windings 322/332, 422/432. However, in some embodiments an interventional element can comprise fewer than three or more than three engagement elements and/or windings. For example, the interventional element 504 shown in FIG. 5 comprises a first end portion 504a, a second end portion 504b, a central longitudinal axis L extending therebetween, and six engagement elements 522. The first end portion 504a can comprise a proximal end portion or a distal end portion of the interventional element 504. The interventional element 504 can comprise a coil formed from a wire or other elongated element wound about the central longitudinal axis L to form six windings 532 corresponding to the engagement elements 522. By including three more windings 532 than the interventional elements 304, 404 of FIGS. 3 and 4, the interventional element 504 may be configured to engage a longer obstruction. The interventional element 504 can include a plurality of end elements 530 at the first end portion 504a of the interventional element 504 and/or a plurality of end elements 530 at the second end portion 504b of the interventional element 504. Only a few of the end elements 530 are labeled in FIG. 5 for ease of illustration. As shown in FIG. 5, in some embodiments the interventional element 504 comprises a similar number of end elements 530 on either side of the engagement elements/windings 522/532.

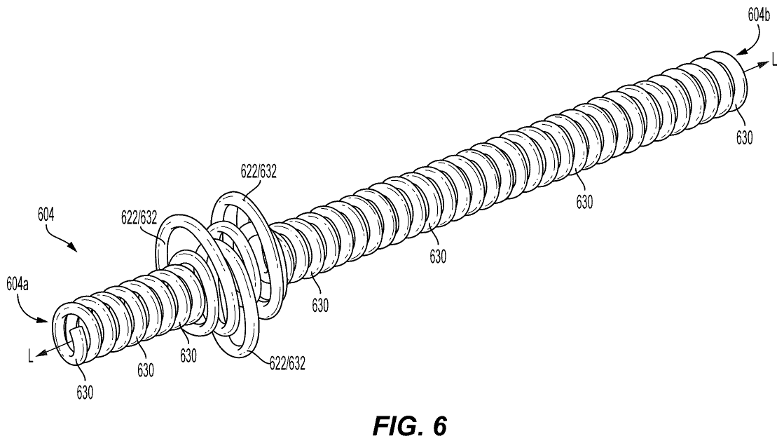

The interventional element 604 shown in FIG. 6 comprises a first end portion 604a, a second end portion 604b, a central longitudinal axis L extending therebetween, and a plurality of engagement elements 622. The first end portion 604a can comprise a proximal end portion or a distal end portion of the interventional element 604. The interventional element 604 can comprise a coil formed from a wire or other elongated element wound about the central longitudinal axis L to form a plurality of windings 632 corresponding to the engagement elements 622. The interventional element 604 can comprise a plurality of end elements 630 (a few of which are labeled in FIG. 6) disposed on either side of the engagement elements/windings 622/632. However, in contrast to the interventional element 504 of FIG. 5, the end elements 630 of interventional element 604 can be asymmetrically distributed on either side of the engagement elements/windings 622/632. For example, the interventional element 604 can comprise fewer end elements 630 at the first end portion 604a of the interventional element 604 than at the second end portion 604b of the interventional element 604. As previously noted, in some embodiments the end elements 630 at the second end portion 604b of the interventional element 604 form the manipulation member. In these embodiments, and others, the interventional element 604 may include many end elements 630 at a proximal end portion of the interventional element 604 to provide sufficient length for navigating the interventional element 604 from an extracorporeal location to a treatment location within a bodily lumen.

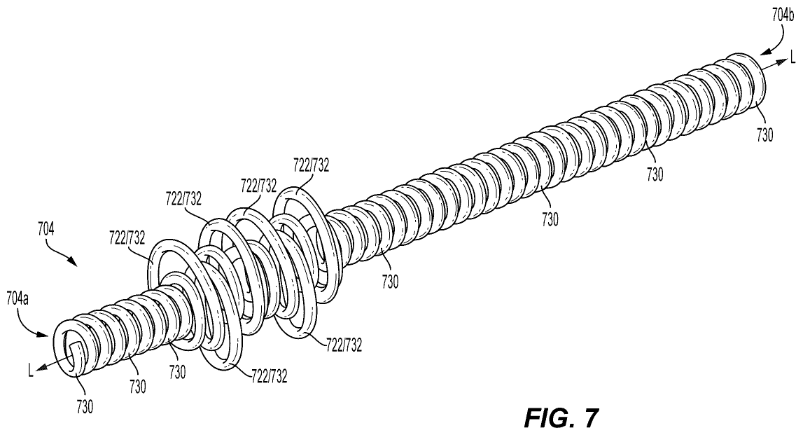

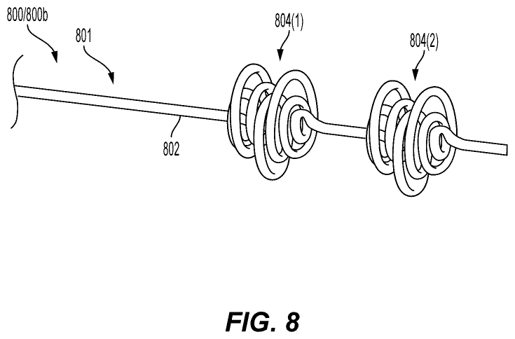

An interventional element of the present technology can include any suitable number or combination of features as disclosed herein. For example, the interventional element 704 shown in FIG. 7 comprises a first end portion 704a, a second end portion 704b, a central longitudinal axis L extending therebetween, six engagement elements 722 (corresponding to six windings 732), a first plurality of end elements 730 at the first end portion 704a (a few of which are labeled in FIG. 7), and a second, greater plurality of end elements 730 at the second end portion 704b (a few of which are labeled in FIG. 7). The first end portion 704a can comprise a proximal end portion or a distal end portion of the interventional element 704.

Although FIG. 7 illustrates one interventional element 704 with six engagement elements 722 adjacent to one another along a length of the interventional element 704, in some embodiments a treatment device can comprise multiple interventional elements 704 spaced apart along a length of the treatment device and/or multiple engagement elements spaced apart along a length of the interventional element and/or treatment device. For example, FIG. 8 depicts a treatment device 801 comprising a manipulation member 802, a first interventional element 804(1) and a second interventional element 804(2) spaced apart from the first interventional element 804(1) along a length of the manipulation member 802. The first interventional element 804(1) and/or the second interventional element 804(2) can comprise three engagement elements and/or windings (as shown in FIG. 8) or any other suitable number of engagement elements and/or windings. The first interventional element 804(1) can have the same number or a different number of engagement elements and/or windings as the second interventional element 804(2). The first interventional element 804(1) can have the same outer diameter or a different outer diameter in the expanded configuration as the second interventional element 804(2). Although not shown in FIG. 8, in some embodiments end elements can extend between the first interventional element 804(1) and the second interventional element 804(2) and/or end elements can extend away from the first interventional element 804(1) and/or the second interventional element 804(2).

In some embodiments, the first interventional element 804(1) and/or the second interventional element 804(2) can be monolithic with the manipulation member 802. For example, the manipulation member 802, first interventional element 804(1), and the second interventional element 804(2) can be formed from a single wire or other elongated element. Still, in some embodiments, the first interventional element 804(1) and/or the second interventional element 804(2) can be mounted on the manipulation member 802. As previously noted, the first interventional element 804(1) and/or the second interventional element 804(2) can be longitudinally and/or rotationally fixed on the manipulation member 802. In some embodiments, the manipulation member 802 carries one or more stops (not shown) that limit longitudinal motion of the first interventional element 804(1) and/or the second interventional element 804(2) relative to the manipulation member 802. In some embodiments, coiled end elements of the first interventional element 804(1) and/or the second interventional element 804(2) can form the manipulation member.

The features of the treatment device 801 can be generally similar to the features of the treatment device 101 of FIGS. 1A-2B. Accordingly, like numbers (e.g., manipulation member 802 versus manipulation member 102, etc.) are used to identify similar or identical components in FIGS. 1A-2B and 8. Any of the features of the treatment device 801 can be combined with each other and/or with the features of the treatment device 101 of FIGS. 1A-2B. Additionally or alternatively, the features of the interventional elements 804(1), 804(2) of treatment device 801 can be similar to any of the interventional elements 304-704 of FIGS. 3-7. Any of the features of the interventional elements 804(1), 804(2) can be combined with each other and/or with the features of the interventional elements 304-704 of FIGS. 3-7.

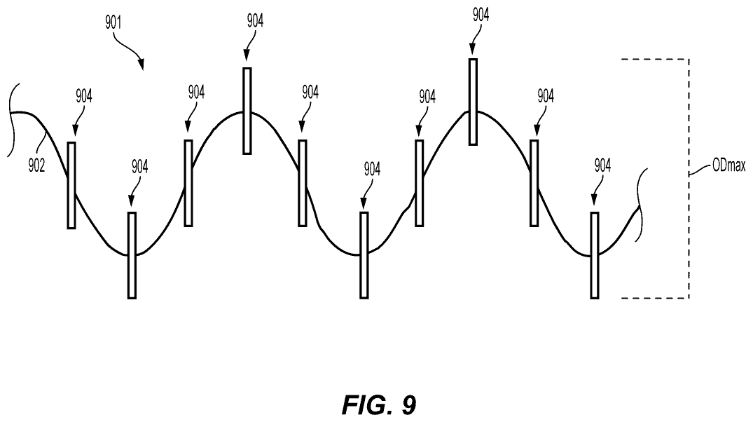

Although FIGS. 1A-8 depict treatment devices 101-108 having generally straight configurations along their lengths, in some embodiments it may be advantageous for a treatment device to be curved along its length. FIG. 9 schematically illustrates such a treatment device 901. As shown in FIG. 9, in some embodiments the manipulation member 902 can undulate, spiral, and/or otherwise curve one or more times along its length. The treatment device 901 can include one or more interventional elements 904 disposed along the length of the manipulation member 902. As previously noted, and as shown in FIG. 9, the interventional elements 904 can be spaced apart along the length of the manipulation member 902. Additionally or alternatively, two or more interventional elements 904 can be located immediately adjacent to one another along the length of the manipulation member 902. Each of the interventional elements 904 can be centered about the manipulation member 902 when in the expanded configuration, or as previously described, eccentrically disposed about the manipulation member 902.

The curved configuration of the manipulation member 902 can facilitate engagement and retrieval of an obstruction within a bodily lumen by distributing interventional elements 904 at various locations throughout the bodily lumen. For example, the interventional elements 904 at the peaks and valleys of the manipulation member 902 shown in FIG. 9 can be configured to engage opposing circumferential portions of the wall of the bodily lumen. The maximum outer diameter ODmax of the device 901 can be greater with a manipulation member 902 that is curved along its length than with a manipulation member 902 that is straight along its length, which can facilitate reaching portions of an obstruction secured to a wall of a bodily lumen.

The manipulation member 902 can be configured to be positioned within a lumen of an elongated shaft in a radially compressed configuration and transitions to a radially expanded once released from the lumen of the elongated shaft. In some embodiments, the manipulation member 902 is substantially straight along its length in the radially compressed configuration and/or is curved along its length to a lesser degree in the radially compressed configuration than in the radially expanded configuration. Alternatively, the manipulation member 902 can remain curved along its length while positioned in the lumen of the elongated shaft.

The features of the treatment device 901 can be generally similar to the features of the treatment device 101 of FIGS. 1A-2B and/or the treatment device 801 of FIG. 8. Accordingly, like numbers (e.g., manipulation member 902 versus manipulation member 102 and/or manipulation member 802, etc.) are used to identify similar or identical components in FIGS. 1A-2B, 8 and 9. Any of the features of the treatment device 901 can be combined with each other and/or with the features of the treatment device 101 of FIGS. 1A-2B and/or the treatment device 801 of FIG. 8. Additionally or alternatively, the features of the interventional elements 904 of treatment device 901 can be similar to any of the interventional elements 304-804 of FIGS. 3-8. Any of the features of the interventional elements 904 can be combined with each other and/or with the features of the interventional elements 304-804 of FIGS. 3-8.

### CONCLUSION

Although many of the embodiments are described above with respect to systems, devices, and methods for restoring or improving patency of blood vessels by removing emboli and/or thrombi, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-9.

As used herein, the terms “distal” and “proximal” define a position or direction with respect to a clinician or a clinician's control device (e.g., a handle of a delivery catheter). For example, the terms, “distal” and “distally” refer to a position distant from or in a direction away from a clinician or a clinician's control device along the length of device. In a related example, the terms “proximal” and “proximally” refer to a position near or in a direction toward a clinician or a clinician's control device along the length of device.

As used herein, the term “longitudinal” can refer to a direction along an axis that extends along a length of a structure, and the term “circumferential” can refer to a direction along an axis that is orthogonal to the longitudinal axis and extends around the circumference of the structure.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method for removing an obstruction from a bodily lumen, the method comprising:
obtaining a treatment system comprising an elongated shaft defining a lumen extending therethrough and a treatment device comprising a manipulation member and an interventional element at a distal portion of the manipulation member, the interventional element comprising a resilient wire formed into a coil having a plurality of windings adjacent to one another along a longitudinal dimension of the interventional element, wherein the interventional element is positioned within the lumen of the elongated shaft at a distal portion of the elongated shaft in a radially compressed configuration in which the windings are circumferentially aligned with one another about the longitudinal dimension;
distally advancing the distal portion of the elongated shaft through the bodily lumen to a treatment location at or adjacent to the obstruction;
releasing the interventional element from the lumen of the elongated shaft such that the interventional element transitions from the radially compressed configuration to a radially expanded configuration in which the windings are circumferentially offset from one another about the longitudinal dimension;
engaging the obstruction with the interventional element in the radially expanded configuration; and
proximally withdrawing the manipulation member to proximally withdraw the interventional element and engaged obstruction from the bodily lumen.

2. The method of claim 1, wherein each of the windings is defined by a perimeter comprising a first region and a second region, and wherein each of the first regions is closer to the central longitudinal axis in the radially compressed state than in the radially expanded state and each of the second regions is farther from the central longitudinal axis in the radially compressed state than in the radially expanded state.

3. The method of claim 2, wherein the first regions are configured to move away from the central longitudinal axis and the second regions are configured to move towards the central longitudinal axis as the interventional element transitions from the radially compressed state to the radially expanded state.

4. The method of claim 2, wherein each of the first regions and each of the second regions extends circumferentially about the central longitudinal axis.

5. The method of claim 2, wherein, when the interventional element is in the radially expanded state, all or a portion of the first region of one of the windings does not circumferentially overlap the first region of another of the windings.

6. The method of claim 1, wherein the plurality of windings comprises three windings angularly offset from one another about a circumference of the interventional element by about 120 degrees.

7. The method of claim 1, wherein the plurality of windings comprises four windings angularly offset from one another about a circumference of the interventional element by about 90 degrees.

8. The method of claim 1, wherein the plurality of windings comprises five windings angularly offset from one another about a circumference of the interventional element by about 72 degrees.

9. The method of claim 1, wherein the resilient wire forms the manipulation member.

10. The method of claim 1, wherein each of the windings defines an opening, and wherein the manipulation member extends through the opening of at least one of the windings.

11. A method for removing an obstruction from a bodily lumen, the method comprising:
obtaining a treatment system comprising an elongated shaft defining a lumen extending therethrough and a treatment device comprising a manipulation member and an interventional element at a distal portion of the manipulation member, the interventional element comprising a resilient wire formed into a coil having a first winding and a second winding adjacent the first winding along a central longitudinal axis of the interventional element, wherein the interventional element is positioned within the lumen of the elongated shaft at a distal portion of the elongated shaft in a radially compressed configuration in which the first and second windings are centered about the central longitudinal axis;
distally advancing the distal portion of the elongated shaft through the bodily lumen to a treatment location at or adjacent to the obstruction;
releasing the interventional element from the lumen of the elongated shaft such that the interventional element transitions from the radially compressed configuration to a radially expanded configuration in which the first and second windings are eccentrically disposed about the central longitudinal axis;
engaging the obstruction with the interventional element in the radially expanded configuration; and
proximally withdrawing the manipulation member to proximally withdraw the interventional element and engaged obstruction from the bodily lumen.

12. The method of claim 11, wherein each of the first and second windings has a first length and second length diametrically opposed to the first length, and wherein, the first lengths are positioned further from the central longitudinal axis in the radially expanded configuration than in the radially compressed configuration and the second lengths are positioned closer to the central longitudinal axis in the radially expanded configuration than in the radially compressed configuration.

13. The method of claim 12, wherein, when the interventional element is in the radially expanded configuration, the first lengths are positioned further from the central longitudinal axis than the second lengths.

14. The method of claim 12, wherein, when the interventional element is in the radially expanded configuration, a maximum outer diameter of the interventional element defined between the first length of the first winding and the first length of the second winding is greater than a local outer diameter of the interventional element defined at any given location along the central longitudinal axis.

15. The method of claim 12, wherein, when the interventional element is in the radially expanded configuration, a maximum outer diameter of the interventional element defined between the first length of the first winding and the first length of the second winding is greater than at least one of a first radial distance defined between the first and second lengths of the first winding or a second radial distance defined between the first and second lengths of the second winding.

16. The method of claim 12, wherein at least one of the first length of the first winding or the first length of the second winding has a greater radius of curvature than the second length of the respective winding.

17. The method of claim 12, wherein at least one of the first length of the first winding or the first length of the second winding has a greater arc length than the second length of the respective winding.

18. The method of claim 11, wherein the resilient wire forms the manipulation member.

19. The method of claim 11, wherein each of the first and second windings defines an opening, and wherein the manipulation member extends through the openings of the first and second windings.
